(19) United States
(12) Patent Application Publication
(10) Pub. No.: US 2012/0046353 A1
(43) Pub. Date: Feb. 23, 2012

(54) CLEISTOCALYX OPERCULATUS-DERIVED COMPOUNDS HAVING INHIBITORY ACTIVITIES AGAINST AVIAN AND SWINE INFLUENZA VIRUSES OR NOVEL INFLUENZA VIRUS

(75) Inventors: Injoong YOON, Daejeon (KR); Eunhee KIM, Daejeon (KR); Hwan-won CHOI, Daejeon (KR); Won Keun OH, Gwangju (KR); Trong Tuan DAO, Nam Dinh (VN); Phuong Thien THUONG, Hanoi (VN)

(73) Assignee: CHOONG ANG VACCINE LAB., Daejeon (KR)

(21) Appl. No.: 12/954,331

(22) Filed: Nov. 24, 2010

Related U.S. Application Data
(63) Continuation of application No. PCT/KR2010/007442, filed on Oct. 28, 2010.

(30) Foreign Application Priority Data
Aug. 17, 2010 (KR) 10-2010-0079391

(51) Int. Cl. A61K 31/353; C07D 311/38; C07C 49/835; A61P 31/16; A01N 43/16; A01N 35/04; A01P 1/00; C07D 311/32; A61K 31/12

(52) U.S. Cl. 514/456; 549/400; 549/403; 568/334; 514/685

(57) ABSTRACT

The present invention relates to a Cleistocalyx operculatus-derived compound having inhibitory activity against neuraminidase and to a composition for preventing and treating diseases caused by avian and swine influenza viruses and novel influenza viruses, comprising the compound as an active ingredient.

### CROSS REFERENCE TO RELATED APPLICATIONS

This application claims priority to International Application No.: PCT/KR2010/007442, with an International Filing Date of Oct. 28, 2010, which claims the benefit of Korean Application No. 10-2010-0079391, filed on Aug. 17, 2010, the entire contents of which are incorporated herein by reference.

### FIELD OF THE INVENTION

The present invention relates to Cleistocalyx operculatus-derived compounds having inhibitory activity against neuraminidase and to a composition for preventing and treating diseases caused by avian and swine influenza viruses and novel influenza viruses, comprising at least one of the compounds as an active ingredient.

More specifically, the present invention relates to fractions, obtained by Cleistocalyx operculatus with ethanol or methanol, saturating the extract with an aqueous solution and fractionating the saturated extract with ethyl acetate, and to neuraminidase inhibitory compounds of Formula 1 purified from the ethyl acetate fractions by chromatography, and also to a composition for preventing and treating diseases related to the neuraminidase of viruses, including influenza, avian and swine influenza viruses and novel influenza viruses, the composition comprising at least one of the above fractions and compounds as an active ingredient.

### DESCRIPTION OF THE PRIOR ART

Influenza viruses are RNA viruses belonging to the Orthomyxoviridae family (Orthomyxovirus) and are classified into three types, A, B and C. Type A can infect a wide range of species, including humans, pigs, other mammals and wild birds, but only humans are infected by types B and C. Avian influenza, swine influenza and novel influenza which have recently become a problem worldwide all belong to type A influenza viruses.

Influenza viruses possess two surface proteins: hemagglutinin (HA) and neuraminidase (NA). Hemagglutinin has 16 subtypes and neuraminidase has 9 subtypes, and thus 144 (15×9) subtypes of influenza viruses can appear. Avian influenza inflection in birds is mainly related to subtypes H5, H7 and H9, and only 3 hemagglutinin subtypes (H1, H2 and H3) and 2 neuraminidase subtypes (N1 and N2) cause influenza infection in humans. Thus, humans are generally not infected by avian influenza. However, in recent years, avian influenza viruses have been transmitted directly to humans without a genetic recombination process.

Avian influenza is transmitted rapidly through poultry, including chickens, ducks and turkeys, as well as wild birds and migratory birds. Avian influenza viruses have spread from Southeast Asia in which outbreaks of avian influenza first occurred. Particularly, avian influenza viruses can also be transmitted through yellow sand from China in the spring season. In Korea, outbreaks of avian influenza, which is an animal viral disease that is becoming a problem worldwide, have occurred since 1966 and caused the slaughter of about 530 million domestic animals, including chickens and ducks, leading to more than 1500 hundred million Won of damage. Also, in Asia, outbreaks of avian influenza have caused the slaughter of more than 2 hundred million domestic animals, causing disastrous damage to the poultry industry.

Swine influenza virus was first reported in 1918. Pigs have both a human influenza receptor (N-acetylneuraminic acid α2,6 galactose) and an avian influenza receptor(N-acetylneuraminic acid α2,3 galactose), and thus are known to serve as mixing vessels for the generation of pandemic influenza viruses through reassortment or adaptation of avian viruses to humans.

Novel influenza (Novel flu) which has been spreading worldwide since its first outbreak in Mexico in 2009 is inferred to be due to mutation of swine influenza virus H1N1 into a form which can pass easily from human to human. Novel influenza generally displays flu-like symptoms, including fever and cough, and there are concerns it could mutate into a more fatal form. According to the report of the World Health Organization (WHO), the number of people infected with novel influenza reached 343,200 up to early October, 2009, and among them, 4000 people died. The general symptoms of novel influenza are very difficult to distinguish from general flu. Novel influenza displays symptoms, including fever, sore throat, cough, snivel and nasal congestion, similar to general flu. According to recent reports, novel influenza is characterized in that it causes severe lung damage compared to the general flu, and thus it is a very important issue to prevent and treat novel influenza. Accordingly, it is necessary to ensure vaccines for preventing novel influenza. However, it is nearly impossible to make vaccines for preventing all RNA viruses in which antigenic drift frequently occur. Thus, in order to cope with outbreaks of avian and swine influenza viruses, novel influenza viruses, or novel pathogens in which avian influenza virus is mixed with novel influenza virus in the worst possible case, it is necessary to develop therapeutic agents capable of preventing and treating infections with such viruses.

Current therapeutic agents against infections with avian and swine influenza virus or novel influenza viruses include Tamiflu (oseltamivir phosphate), a neuraminidase inhibitor that is administered orally, and Relenza (zanamivir), a neuraminidase inhibitor that is inhaled by mouth. Tamiflu which is frequently used as an oral medication was reported to have side effects, including nausea, vomiting, and abnormalities of the nervous or mental system. In Japan, 15 infant deaths caused by Tamiflu were confirmed, suggesting that Tamiflu has serious side effects. Also, outbreaks of Tamiflu-resistant virus must be coped with, but the occurrence of Tamiflu-resistant virus was already reported and even the human-to-human transmission of Tamiflu-resistant virus was reported. Thus, the development of therapeutic agents against avian and swine influenza viruses and novel influenza viruses is necessary for human health and safety.

With respect to the process of viral infection and replication, a virus infects a cell, and then makes new viruses in the cell. For the transmission of the viruses to other cells, the cell surface sialic acid needs to be cleaved with the viral neuraminidase. It is known that Tamiflu-resistant avian influenza virus usually occurs through three genetic mutations (Arg292Lys, Asn274Ser and His294Tyr) that change the amino acids of the Tamiflu receptor. Tamiflu-resistant avian influenza viruses having a His-to-Tyr mutation at amino acid position 294 occur because the hydrophobic residue of Tamiflu cannot bind to the Tamiflu receptor. This Tamiflu-resistant virus was found at a rate of 10-25% in USA and Europe during the years 2007-2008 and increases the possibility of mutation of novel influenza viruses. Thus, the appearance of such Tamiflu-resistant viruses suggests that humans should continuously develop novel therapeutic agents against viruses.

In the present invention, in view of the urgency of defense against avian and swine influenza viruses and novel influenza viruses, neuraminidase inhibitors against avian and swine influenza viruses and novel influenza viruses were investigated on foods and medicinal plants which would be applied immediately after the confirmation of effects thereof. Generally, in order to develop novel drugs, it is more advantageous to find novel active ingredients from natural medicinal materials which are used in traditional medicine, compared to experimentally modifying conventional drugs. Particularly, because such active ingredients have been used for a long time, they have a low risk of toxicity. This can be demonstrated from the fact that the active ingredient of Tamiflu is obtained from the fruit of star anise, a Chinese native plant.

Cleistocalyx operculatus which is used in the present invention is a plant that is distributed over an area ranging from tropical Asia to the northern part of Austria and grows to a height of 6-12 m. It has been traditionally used as a drug for diseases of the digestive system in Vietnam or the western part of China, and the bud of Cleistocalyx operculatus is called “nu voi” and has been mainly used to inhibit shock or lower fever (Loi, D. T. Medical Publishing House, Hanoi, Vietnam, 2001, 423-424). Cleistocalyx operculatus was reported to be effective for the treatment of liver-related diseases or diabetes (Lu, Y. H. et. al, Zhongguo Zhong Yao ZaZhi, 2003, 28, 964-966; Mai, T. T. et. al, Biosci. Biotechnol. Biochem. 2007, 71, 69-76) and is known to contain large amounts of antioxidant or anti-inflammatory compounds (Min, B. S. et. al, Chem. Pharm. Bull, 2008, 56, 1725-1728; Dung, N. T. et. al, Food Chem. Toxicol. 2009, 47, 449-453). In addition, it was recently found that an ethanol extract of the bud of Cleistocalyx operculatus has antimicrobial effects (Dung, N. T. et. al. Food Chem. Toxicol, 2008, 46, 3632-3639.).

The present inventors have purified compounds from an organic solvent extract of Cleistocalyx operculatus and have compared the activities of the compounds against the neuraminidases (His294Tyr) of avian and swine influenza viruses, novel influenza viruses and Tamiflu-resistant novel influenza viruses. As a result, the present inventors have found that the compounds of the present invention have inhibitory activities against avian and swine influenza viruses and novel influenza viruses, thereby completing the present invention.

### SUMMARY OF THE INVENTION

It is an object of the present invention to provide a composition containing, as an active ingredient, one or a mixture of two or more of compounds, isolated and purified from Cleistocalyx operculatus, which show preventive and therapeutic effects on avian influenza- and novel influenza-related diseases by inhibiting the neuraminidases of avian and swine influenza viruses, novel influenza viruses and Tamiflu-resistant novel influenza viruses.

The present inventors collected a variety of native plants and medicinal herbal plants and either cultured avian and swine influenza viruses or cloned the neuraminidases of novel influenza viruses and Tamiflu-resistant novel influenza viruses. Then, the cultured viruses or cloned neuraminidases were introduced into cells together with the collected plants in order to examine the inhibitory activities of the plants against the neuraminidase enzymes. As a result, Cleistocalyx operculatus was selected as a candidate plant. Then, Cleistocalyx operculatus was extracted with organic solvent alcohol (e.g., methanol, ethanol or propanol) or water-soluble alcohol and subjected to organic solvent/water partition, column chromatography, conventional processes used for the isolation and extraction of plant components, or a combination of two or more thereof. The crude extract can be further purified, if necessary.

In the present invention, Cleistocalyx operculatus was extracted with alcohol, and compounds were isolated and purified from the extract using chromatography. The chemical structures and physical and chemical properties of the compounds were analyzed to confirm the structures thereof. As a result, it was found that, among the compounds, compounds represented by the following formula 1 have inhibitory activity against viral neuraminidase and Tamiflu-resistant neuraminidase, thereby completing the present invention:

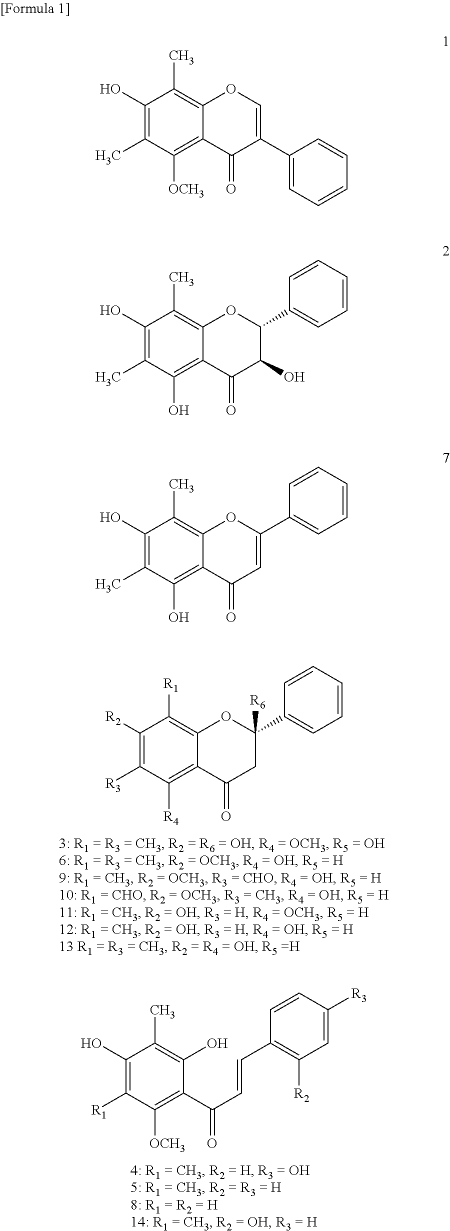

The present invention provides a composition containing, as an active ingredient, at least one compound of the above-described compounds, which has preventive and therapeutic effects on avian and swine influenza virus- and novel influenza virus-related diseases by inhibiting the neuraminidase of the viruses.

The activities of the above-described compounds were confirmed by a method comprising the steps of: isolating and purifying compounds from an organic solvent extract of Cleistocalyx operculatus; examining the physical structures and physical and chemical properties of the purified compounds; making the neuraminidase of novel influenza virus and the neuraminidase (His294Tyr) of Tamiflu-resistant novel virus; and measuring the inhibitory activities of the compounds against the neuraminidase of avian influenza virus (H9N2) and swine influenza virus (H1N1) and the neuraminidase of novel influenza virus and Tamiflu-resistant novel influenza virus.

The inventive inhibitory compounds against the neuraminidase of avian influenza virus (H9N2) and swine influenza virus (H1N1) and the neuraminidase of novel influenza virus and Tamiflu-resistant novel influenza virus can be easily obtained by extracting Cleistocalyx operculatus with an organic solvent (e.g., alcohol, aqueous alcohol solution, ethyl acetate, ether, acetone, chloroform, etc.) and subjecting the extract to one or more of conventional methods used for the isolation and purification of plant components, including hexane/water partition, a process utilizing adsorption resin such as HP-20 resin, and a process with column chromatography. The crude extract may, if necessary, be further purified by phase separation.

Chromatographic processes that may be used in the present invention include silica gel column chromatography, LH-20 column chromatography, thin layer chromatography (TLC) and high-performance liquid chromatography.

The Cleistocalyx operculatus extract according to the present invention and compounds isolated thereform have high stability and thus can be used as additives to foods or drugs.

Dosage forms of a pharmaceutical composition containing the extract or compounds of the present invention include oral formulations such as powders, granules, tablets, capsules, suspensions, emulsions, syrups and aerosols, and parenteral formulations such as external preparations, suppositories, and sterile injectable solutions. The composition may be formulated using any appropriate method known in the art. Examples of carriers, excipients or diluents that may be included in the composition according to the present invention include lactose, dextrose, sucrose, sorbitol, mannitol, xylitol, erythritol, maltitol, starch, acacia ruber, alginate, gelatin, calcium phosphate, calcium silicate, cellulose, methyl cellulose, microcrystalline cellulose, polyvinyl pyrrolidone, water, methyl hydroxybenzoate, propyl hydroxylbenzoate, talc, magnesium stearate and mineral oil. The inventive composition may be formulated with generally used diluents or excipients, such as fillers, extenders, binders, wetting agents, disintegrants and surfactants. Solid formulations for oral administration include tablets, pills, powders, granules and capsules. These solid formulations are prepared by mixing the extract with at least one excipient, for example, starch, calcium carbonate, sucrose, lactose and gelatin. Also, in addition to simple excipients, lubricants such as magnesium stearate or talc are also used. Liquid preparations for oral administration include suspensions, solutions, emulsions, syrups, etc., and may include commonly used, simple diluents such as water and liquid paraffin, and, if desired, may further include various excipients, for example, humectants, sweeteners, aromatics and preservatives. Formulations for parenteral administration include sterile aqueous solutions, nonaqueous solvents, suspensions, emulsions, freeze-dried preparations, and suppositories. As the nonaqueous solvents and the suspensions, propylene glycol, polyethylene glycol, vegetable oil such as olive oil, injectable ester (such as ethyl oleate), etc. may be used. As a base for the suppository, witepsol, macrogol, tween 61, cacao butter, laurin butter, glycerogelatin, etc. may be used.

The dosage of the extract or compound of the present invention may vary depending on the age, sex and body weight of the subject in need of treatment, the particular disease or condition to be treated, the severity of the disease or condition, administration route, or the prescriber's decision. The determination of the dosage considering these factors will be easily understood by those skilled in the art. The dosage is generally 0.01 mg/kg/day-2000 mg/kg/day, and preferably 0.1 mg/kg/day-500 mg/kg/day. The composition of the present invention may be administered once or several times a day. The dosage does not limit the scope of the present invention in any way. The extract or compound of the present invention may be administered to mammals, including rats, domestic animals and humans, by various routes. All the routes of administration may be expected, and for example, the extract or compound of the present invention may be administered by oral, intrarectal, intravenous, intramuscular, subcutaneous, intrathecal or intracerebroventricular injections. The extract or compound of the present invention has little or no toxicity or side effects, and thus can be used for preventative purposes without fear even in long-term administration.

The present invention also provides a health functional food for preventing diseases caused by avian and swine influenza viruses and novel influenza viruses, the food comprising the Cleistocalyx operculatus extract and an acceptable food additive. The health functional food of the present invention may be used in the form of tablets, capsules, pills or liquids. Foods to which the extract of the present invention may be added include, for example, dairy products, including drinks, meats, sausages, bread, candies, snacks, noodles, and ice creams; beverages, including soups and ion beverages, and nutrition supplement products, including alcoholic beverages or vitamin complexes. Specifically, the present invention provides a health functional food for prevention and treatment of novel influenza-associated diseases, the food comprising the Cleistocalyx operculatus extract and an acceptable food additive.

The present invention also provides feed additives and therapeutic agents for prevention and treatment of animal diseases, which comprise the Cleistocalyx operculatus extract and a Cleistocalyx operculatus-derivedx compound. The feed additive and therapeutic agent of the present invention may be used for neuraminidase-related zoonotic diseases such as infectious enteritis. Particularly, the present invention provides animal feed additives and animal drugs for preventing and treating diseases associated with novel influenza virus and avian and swine influenza viruses.

### EFFECT OF THE INVENTION

The Cleistocalyx operculatus extract and Cleistocalyx operculatus-derived compound according to the present invention have the therapeutic effect of noncompetitively inhibiting the neuraminidase of avian and swine influenza viruses and the neuraminidase of Tamiflu-resistant novel influenza viruses.

Furthermore, when avian influenza (H9N2) and swine influenza (H1N1) viruses were treated with a combination of Tamiflu with the compound of the present invention, the amount of Tamiflu used could be reduced. Namely, when avian influenza (H9N2) and swine influenza (H1N1) viruses were treated with a combination of Tamiflu with the compound of the present invention were treated with Tamiflu alone without compound 4 of the present invention, Tamiflu showed ID₅₀ values of 4.24±0.82 ng/ml and 39.04±1.03 ng/ml, but when avian influenza (H9N2) and swine influenza (H1N1) viruses were treated with Tamiflu in combination with 1 μg/ml of compound 4, Tamiflu showed ID₅₀ values of 0.46±0.42 ng/ml and 10.51±0.64 ng/ml, suggesting that compound 4 increased the inhibitory activity of Tamiflu by about 10 times against avian influenza (H9N2) virus and about 4 times against swine influenza (H1N1) virus.

Accordingly, the Cleistocalyx operculatus extract according to the present invention and the compounds isolated therefrom can have preventive and therapeutic effects on avian influenza- and novel influenza-related diseases by the neuraminidases of avian and swine influenza viruses, novel influenza viruses and Tamiflup-resistant novel influenza viruses. Also, they have low cytotoxicity, and thus can be very advantageously used in drugs, cosmetics, heath foods, animal feeds, animal drugs, etc.

### BRIEF DESCRIPTION OF THE DRAWINGS

FIG. 1 is a spectrum showing the results of HPLC analysis for the peaks and structures of compounds isolated from Cleistocalyx operculatus ;

FIG. 2 is Lineweaver Burk plots showing that, among the compounds isolated from Cleistocalyx operculatus, compound 4 showing strong inhibitory activity is a non-competitive inhibitor;

FIG. 3 is Lineweaver Burk plots showing that, among the compounds isolated from Cleistocalyx operculatus, compound 5 showing strong inhibitory activity is a non-competitive inhibitor;

FIG. 4 is Lineweaver Burk plots showing that, among the compounds isolated from Cleistocalyx operculatus, compound 8 showing strong inhibitory activity is a non-competitive inhibitor;

FIG. 5 is Lineweaver Burk plots showing that, among the compounds isolated from Cleistocalyx operculatus, compound 14 showing strong inhibitory activity is a non-competitive inhibitor;

FIG. 6 shows the inhibitory activity of compound 4 (1 μg/ml) against the neuraminidase of swine influenza virus according to Tamiflu concentration (ng/ml); and

FIG. 7 shows the inhibitory activity of compound 4 (1 μg/ml) against the neuraminidase of avian influenza virus according to Tamiflu concentration (ng/ml).

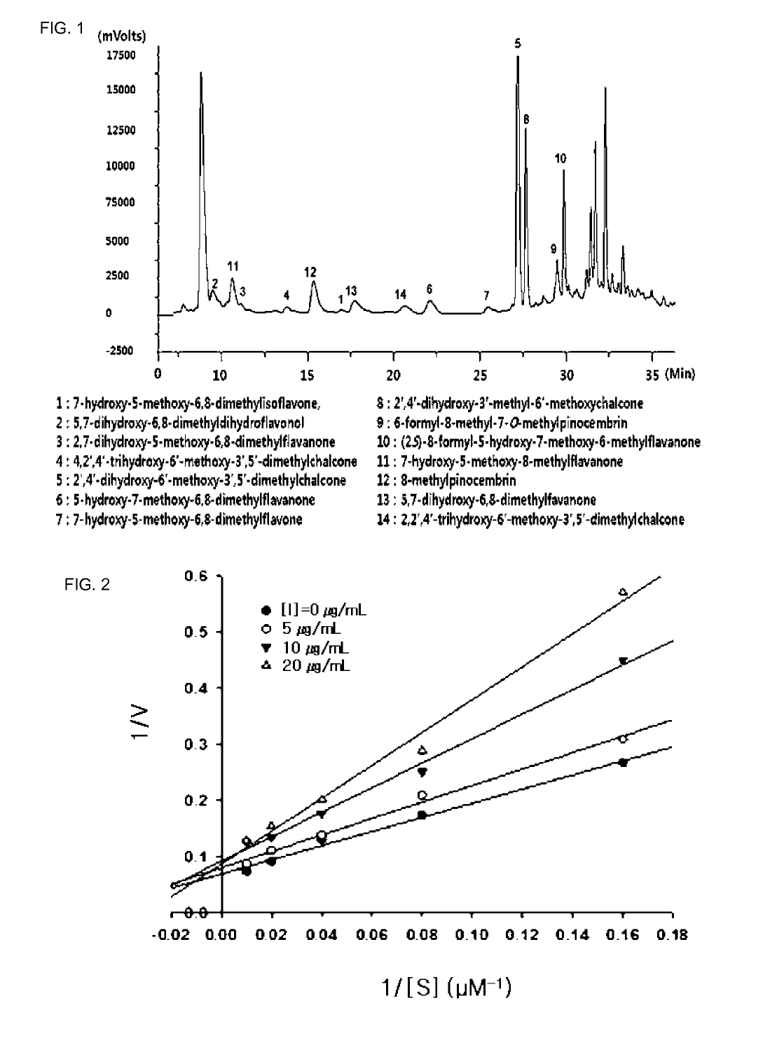

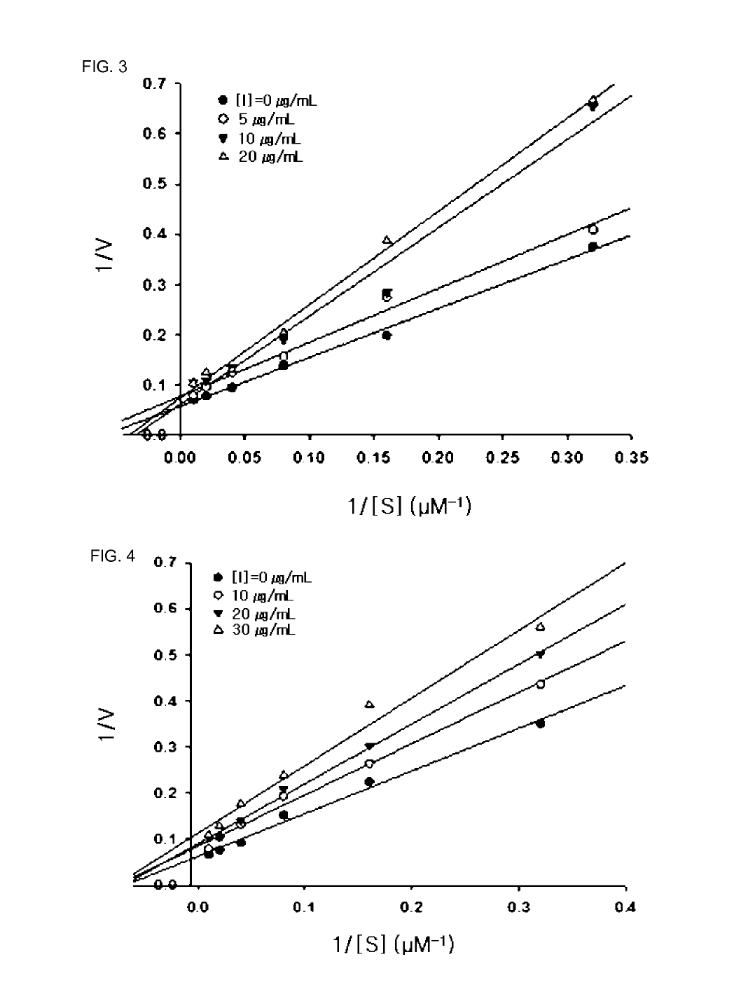

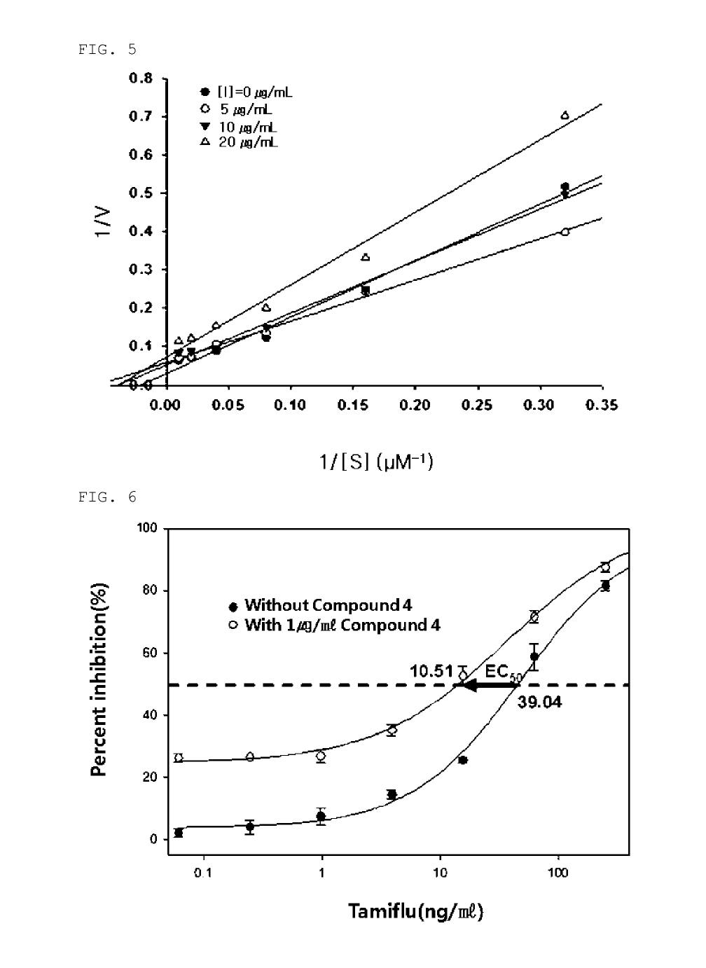

### DETAILED DESCRIPTION OF THE PREFERRED EMBODIMENTS

Hereinafter, examples of the present invention will be described in detail. However, the present invention is not limited to the examples set forth herein and can be embodied in other forms. Rather, the examples set forth herein are provided so that the disclosure will be thorough and complete, and will fully convey the scope of the invention to those skilled in the art.

### Example 1

### Examination of Conditions of Solvent Extraction of Active Compounds from Cleistocalyx Operculatus

In order to compare the degrees of extraction of active compounds from Cleistocalyx operculatus with distilled water, 30-100% ethanol aqueous solution, methanol, acetone, ethyl acetate and chloroform solvents, 100 g of the dried bud of Cleistocalyx operculatus was ultrasonically extracted three times with 500 ml of each of the solvents for 2 hours, and then each extract was concentrated under reduced pressure. The amounts of the extracts were compared and, as a result, an amount ranging from 4.2 g (acetone) to 15.7 g (distilled water) was shown. Then, in order to compare the activities of the extracts against neuraminidase, the activity of each extract was measured at a final extract concentration of 20 μg/Ml using a viral culture of Example 4-2 according to a method of Example 6. As can be seen from the results shown in Table 1 below, the 50-100% ethanol extracts and the methanol extract showed good activity. However, the 70% ethanol extract was selected as the optimum extraction condition as a result of comparing the extraction solvents acceptable to the human body, and the amounts and activities of the extracts for the solvents.

TABLE 1

| | | Inhibitory activity |
|---|---|---|
| | | against neuraminidase of |
| | Amount of | swine influenza virus |
| Condition | extract | (20 μg/Ml) |

| | | | |
|---|---|---|---|
| 30% methanol | 16.0 | g | 19.3% |
| 50% ethanol | 14.1 | g | 36.9% |
| 70% ethanol | 12.2 | g | 51.5% |
| 90% ethanol | 10.7 | g | 44.5% |
| Ethanol | 8.8 | g | 40.1% |
| Methanol | 11.3 | g | 42.8% |
| Acetone | 4.2 | g | ≦5% |
| Ethyl acetate | 5.4 | g | 32.4% |
| Chloroform | 4.5 | g | ≦15% |
| Distilled water | 15.7 | g | ≦5% |

### Example 2

### Isolation of Solvent Extracts and Compounds from Cleistocalyx operculatus

1.5 kg of dried Cleistocalyx operculatus was ultrasonically extracted three times with 10 l of 70% ethanol for 4 hours. The 70% ethanol extract (hereinafter referred to “fraction A”) was concentrated under reduced pressure, and 170 g of the concentrate was suspended in water (2 l) and was fractionated sequentially into an n-hexane (2 l) extract (hereinafter referred to “fraction B”), an ethyl acetate (2 l) extract (hereinafter referred to as “fraction C”) and a butanol (2 l) extract (hereinafter referred to as fraction D″). Then, the inhibitory activities of each of the solvent fractions against the neuraminidases of avian influenza virus and swine influenza virus were measured. As a result, the ethyl acetate fraction showed strong activity.

75 g of the ethyl acetate fraction was subjected to silica gel column chromatography with a solvent gradient of hexane-acetone 4:1→0:1, thereby obtaining 9 fractions (fractions 1 to 9). Among these fractions, 2.5 g of fraction 3 showing strong activity was loaded onto Sephadex LH-20) resin and eluted with methanol, thereby obtaining 750 mg of compound 5 (2′,4′-dihydroxy-6′-methoxy-3′,5′-dimethylchalcone). Fraction 4 (4.2 g) was subjected to reverse phase (RP-18) column chromatography using a mixed solvent of MeOH—H₂O at a solvent gradient of 2:1→10:1, thereby obtaining 5 sub-fractions (fractions 4.1 to 4.5). Sub-fraction F4.2 (160 mg) was HPLC [YMC Pak C₁₈ column 10×250 mm; 10 μm particle size; 2 Ml/min; UV detection: 205-254 nm] using MeOH—H₂O (0-65 min: 63% MeOH, 65-70 min: 100% MeOH, 70-80 min: 100% MeOH), thereby obtaining compound 1 (7-hydroxy-5-methoxy-6,8-dimethylisoflavone, t_{R}=45.0 min, 3.5 mg), compound 6 (5-hydroxy-7-methoxy-6,8-dimethylflavanone, t_{R}=49.0 min, 13.5 mg), and compound 7 (7-hydroxy-5-methoxy-6,8-dimethylflavone, t_{R}=63.0 min, 4.5 mg). Sub-fraction F4.3 (220 mg) was subjected to HPLC using MeOH—H₂O (0-55 min: 65% MeOH, 60 min: 100% MeOH) under the same conditions as the case of sub-fraction F3.2, thereby obtaining compound 8 (2′,4′-dihydroxy-3′-methyl-6′-methoxychalcone, t_{R}=31.0 min, 19.5 mg), compound 9 (6-formyl-8-methyl-7-O-methylpinocembrin, t_{R}=45.0 min, 9.5 mg) and compound 10 [(2S)-8-formyl-5-hydroxy-7-methoxy-6-methylflavanone, t_{R}=48.0 min, 14.0 mg].

Fraction F5 was loaded onto Sephadex LH-20 resin and then eluted with methanol, thereby obtaining 4 sub-fractions (fractions 5.1-5.5). Sub-fraction F5.3 was subjected to reverse phase (RP-18) column chromatography using a mixed solvent of MeOH—H₂O with a solvent gradient of 1:1→10:1, thereby obtaining 5 sub-fraction (F5.3.1 to F5.3.5). Sub-fraction F5.3.2 (150 mg) was subjected to HPLC [YMC Pak C₁₈ column 20×150 mm; 4 μm particle size, 3 Ml/min; UV detection: 254 nm] using MeOH—H₂O (0-45 min: 58% MeOH, 50 min: 100% MeOH), thereby obtaining compound 2 (5,7-dihydroxy-6,8-dimethyldihydroflavonol, t_{R}=28.0 min, 4.5 mg) and compound 11 (7-hydroxy-5-methoxy-8-methylflavanone, t_{R}=40.0 min, 14 mg).

Sub-fraction F5.3.3 (170 mg) was subjected to HPLC [YMC Pak C₁₈ column 20×150 mm; 4 μm particle size; 3 Ml/min; UV detection: 254 nm] using MeOH—H₂O (0-65 min: 62% MeOH, 70 min: 100% MeOH), thereby obtaining compound 3 (2,7-dihydroxy-5-methoxy-6,8-dimethylflavanone, t_{R}=33.0 min, 3.0 mg), compound 12 (8-methylpinocembrin, t_{R}=42.0 min, 8.5 mg), and compound 13 (5,7-dihydroxy-6,8-dimethylflavanone, t_{R}=56.0 min, 15.5 mg).

Finally, sub-fraction F5.3.4 (110 mg) was subjected to HPLC [YMC Pak C₁₈ column 20×150 mm; 4 μm particle size, 3 Ml/min; UV detection: 254 nm] using MeOH—H₂O (0-45 min: 65% MeOH, 50 min: 100% MeOH), thereby obtaining compound 4 (4,2′,4′-trihydroxy-6′-methoxy-3′,5′-dimethylchalcone, t_{R}=29.0 min, 6.5 mg) and compound 14 (2,2′,4′-trihydroxy-6′-methoxy-3′,5′-dimethylchalcone, t_{R}=37.5 min, 9.5 mg).

The activities of the above compounds against neuraminidase were measured according to the methods of Examples 6 and 7, and the results of the measurement are shown in Tables 2 and 3 below.

### Example 3

### Analysis of Physical and Chemical Properties and Chemical Structures of Compounds Derived From Cleistocalyx operculatus

The structures of the compounds from Cleistocalyx operculatus in Example 2 above were analyzed. The chemical structures of the compounds were analyzed on the basis of the molecular weights obtained by an electrospray Ionization mass spectrometer and the results of ¹H and ¹³C-NMR analysis.

As a result, the isolated compounds had the structures shown in Formula 1, and the chemical properties and results of ¹H and ¹³C-NMR analysis of the compounds are summarized in Tables below.

Compound 1 (7-Hydroxy-5-methoxy-6,8-dimethylisoflavone): yellow amorphous powder; UV (MeOH) λmax nm (log ε): 255 (4.32), 298 (3.79); IR (KBr) νₘₐₓ 3386 (OH), 2926, 1637 (C═O), 1591, 1447, 1230, 1136 cm⁻¹; EIMS m/z (rel.int.): 296[M]⁺, 100, 295(31), 281(89), 278(57), 265(22), 250(17), 195(18), 77 (19); HREIMS m/z 296.1047 [M]⁺ (calcd for C₁₈H₁₆O₄, 296.1049); ¹H-NMR (CD₃COCD₃, 500 MHz) δ 7.88 (1H, s, H-2), 7.51 (2H, d, J=8.4 Hz, H-2′, H-6′), 7.34 (3H, m, H-3′, H-4′, H-5′), 3.81 (3H, s, 5-OCH₃), 2.28 (3H, s, 8-CH₃), 2.23 (3H, s, 6-CH₃); ¹³C-NMR (CD₃COCD₃, 125 MHz) δ 175.4 (C═O), 156.7 (C-7), 156.3 (C-5), 154.9 (C-9), 151.0 (C-2), 132.2 (C-1′), 129.2 (C-2, C-6), 128.4 (C-3′, C-5′), 127.9 (C-4′), 125.7 (C-3), 115.5 (C-6), 113.1 (C-10), 106.9 (C-8), 61.7 (5-OCH₃), 8.2 (6-CH₃), 8.1 (8-CH₃).

Compound 2 (5,7-Dihydroxy-6,8-dimethyldihydroflavonol): brown amorphous powder; [a]_{D}²⁶-24.0° (c 0.08, MeOH); UV (MeOH) λmax nm (log ε): 297 (4.45), 340 (3.84); IR (KBr) νₘₐₓ 3423 (OH), 2927, 1639 (C═O), 1469, 1282, 1123 cm⁻¹; EIMS m/z (rel.int.): 300[M]⁺, 67, 271(14), 181(100), 152(49), 77 (10); HREIMS m/z 300.0999 [M]⁺ (calcd for C₁₇H₁₆O₅, 300.0998); ¹H-NMR (CD₃COCD₃, 500 MHz) δ 7.56 (2H, d, J=8.0 Hz, H-2′, H-6′), 7.41 (3H, m, H-3′, H-4′, H-5′), 5.04 (1H, d, J=11.0 Hz, H-2), 4.51 (1H, d, J=11.0 Hz, H-3), 2.02 (3H, s, 8-CH₃), 1.97 (3H, s, 6-CH₃); 13C-NMR (CD₃COCD₃, 125 MHz) δ 198.9 (C═O), 164.7 (C-7), 160.3 (C-9), 158.9 (C-5), 139.1 (C-2), 129.9 (C-4′), 129.6 (C-3′, C-5′), 129.0 (C-2′, C-6′), 105.5 (C-8), 104.5 (C-6), 101.8 (C-10), 85.0 (C-2), 74.1 (C-3), 8.1 (8-CH₃), 7.6 (6-CH₃).

Compound 3 (2,7-Dihydroxy-5-methoxy-6,8-dimethylflavanone): brown amorphous powder; UV (MeOH) λmax nm (log ε): 294 (4.47), 338 (3.87); IR (KBr) νₘₐₓ 3391 (OH), 2926, 1681 (C═O), 1621, 1410, 1338, 1114 cm⁻¹; EIMS m/z (rel.int.); 314[M]⁺, 24, 223(77), 195(100), 152(17), 77 (8); HREIMS m/z 314.1152 [M]⁺ (calcd for C₁₈H₁₈O₅, 314.1154); ¹H-NMR (CD₃COCD₃, 500 MHz) δ 7.29 (2H, d, J=8.0 Hz, H-2′, H-6′), 7.25 (2H, m, H-3′, H-5′), 7.23 (1H, m, H-4′), 3.97 (3H, s, 5-OCH₃), 3.24 (1H, d, J=13.5 Hz, H-3 eq), 3.16 (1H, d, J=13.5 Hz, H-3ax), 2.09 (3H, s, 8-CH₃), 2.03 (3H, s, 6-CH₃); ¹³C-NMR (CD₃COCD₃, 125 MHz) S 193.7 (C═O), 167.9 (C-9), 162.3 (C-7), 155.2 (C-5), 132.9 (C-1′), 130.6 (C-2′, C-6′), 128.3 (C-3′, C-5′), 127.4 (C-4′), 109.3 (C-6), 104.8 (C-10), 103.8 (C-2), 101.6 (C-8), 61.7 (5-OCH₃), 42.0 (C-3), 7.9 (8-CH₃), 7.2 (6-CH₃).

Compound 4 (4,2′,4′-Trihydroxy-6′-methoxy-3′,5′-dimethylchalcone): yellow amorphous powder; UV (MeOH) λmax nm (log ε): 298 (3.91), 362 (4.47); IR (KBr) νₘₐₓ 3385 (OH), 2931, 1605 (C═O), 1545, 1437, 1229, 1164 cm¹; 314[M]⁺, 78, 313(18), 221(10), 194(100), 166(19), 136 (20); HREIMS m/z 314.1156 [M]⁺ (calcd for C₁₈H₁₈O₅, 314.1154); ¹H-NMR (CD₃COCD₃, 500 MHz) δ 13.96 (1H, s, 2′-OH), 7.92 (1H, d, J=15.5 Hz, H-α), 7.82 (1H, d, J=15.5 Hz, H-β), 7.65 (2H, d, J=8.5 Hz, H-2, H-6), 6.94 (2H, d, J=8.5 Hz, H-3, H-5), 3.69 (3H, s, 6′-OCH₃), 2.15 (3H, s, 5′-CH₃), 2.09 (3H, s, 3′-CH₃); ¹³C-NMR (CD₃COCD₃, 125 MHz) δ 193.8 (C═O), 162.7 (C-2′), 161.4 (C-4′), 160.7 (C-4), 159.8 (C-6′), 144.3 (C-β), 131.4 (C-2, C-6), 128.1 (C-1), 124.4 (C-α), 116.9 (C-3, C-5), 110.6 (C-5′), 109.2 (C-1′), 107.9 (C-3′), 62.6 (6′-OCH₃), 9.0 (5′-CH₃), 8.3 (3′-CH₃).

Compound 5 (2′,4′-Dihydroxy-6′-methoxy-3′,5′-dimethylchalcone): orange needles; UV (MeOH) λmax: 205, 340 nm; EI-MS m/z 298 [M]⁺, 221, 206, 194, 166, 131, 103, 91, 77; ¹H-NMR (CD₃COCD₃, 500 MHz) δ 13.82 (1H, s, 2′-OH), 8.05 (1H, d, J=16.0 Hz, H-β), 7.82 (1H, d, J=16.0 Hz, H-α), 7.73 (2H, d, J=8.0 Hz, H-2, H-6), 7.45 (3H, m, H-3, H-4, H-5), 3.68 (3H, s, 6′-OCH₃), 2.16 (3H, s, 5′-CH₃), 2.11 (3H, s, 3′-CH₃); ¹³C-NMR (CD₃COCD₃, 125 MHz) δ 193.9 (C═O), 162.8 (C-2′), 161.8 (C-4′), 159.9 (C-6′), 143.4 (C-β), 136.4 (C-1), 131.2 (C-4), 129.9 (C-3, C-5), 127.8 (C-α, C-2, C-6), 110.7 (C-1′), 109.2 (C-5′), 107.9 (C-3′), 62.4 (6′-OCH₃), 9.0 (5′-CH₃), 8.3 (3′-CH₃).

Compound 7 (7-Hydroxy-5-methoxy-6,8-dimethylfavanone): yellow powder ¹H-NMR (CDCl₃, 500 MHz) δ 7.90 (2H, m, H-2′, H-6′), 7.52 (3H, m, H-3′, H-4′, H-5′), 6.71 (1H, s, H-3), 3.87 (3H, s, 5-OCH₃), 2.44 (3H, s, 8-CH₃), 2.26 (3H, s, 6-CH₃); ¹³C-NMR (CDCl₃, 125 MHz) δ 177.8 (C═O), 160.9 (C-2), 156.9 (C-7), 155.8 (C-5), 154.9 (C-9), 131.9 (C-1′), 131.2 (C-4′), 129.0 (C-3′, C-5′), 126.0 (C-2′, C-6′), 115.3 (C-6), 112.3 (C-10), 108.2 (C-3), 107.2 (C-8), 61.8 (5-OCH₃), 8.6 (8-CH₃), 8.3 (6-CH₃).

Compound 8 (2′,4′-Dihydroxy-3′-methyl-6′-methoxychalcone): yellow oil; UV (MeOH) λmax: 310, 348 nm; EI-MS m/z 284 [M]⁺, 207, 181, 122, 77; ¹H-NMR (CD₃OD, 500 MHz) δ 8.01 (1H, d, J=15.5 Hz, H-β), 7.78 (1H, d, J=15.5 Hz, H-α), 7.67 (2H, d, J=8.5 Hz, H-2, H-6), 7.42 (3H, m, H-3, H-4, H-5), 6.17 (1H, s, H-5′), 3.68 (3H, s, 6′-OCH₃), 2.08 (3H, s, 3′-CH₃); ¹³C-NMR (CD₃OD, 125 MHz) δ 194.4 (C═O), 165.4 (C-4′), 165.3 (C-2′), 162.8 (C-6′), 143.9 (C-(3), 136.9 (C-1), 131.5 (C-4), 130.2 (C-2, C-6), 129.5 (C-3, C-5), 128.0 (C-α), 112.3 (C-1′), 109.7 (C-3′), 99.0 (C-5′), 62.7 (6′-OCH₃), 8.6 (3′-CH₃).

Compound 9 (6-Formyl-8-methyl-7-O-methylpinocembrin): orange needles; UV (MeOH) λmax: 258, 345 nm; EI-MS m/z 312 [M]⁺, 297, 284, 235, 208, 207, 180, 152, 104, 77; ¹H-NMR (CDCl₃, 500 MHz) δ 10.23 (1H, s, 6-CHO), 7.45 (2H, m, H-2′, H-6′), 7.42 (3H, m, H-3′, H-4′, H-5′), 5.54 (1H, dd, J=12.5, 2.5 Hz, H-2), 3.91 (3H, s, 7-OCH₃), 3.07 (1H, dd, J=17.0, 12.5 Hz, H-3), 2.88 (1H, dd, J=17.0, 2.5 Hz, H-3), 2.09 (3H, s, 8-CH₃); ¹³C-NMR (CDCl₃, 125 MHz) δ 192.7 (6-CHO), 187.4 (C═O), 167.9 (C-5), 166.3 (C-7), 165.1 (C-9), 137.7 (C-1′), 129.1 (C-4′), 128.9 (C-3′, C-5′), 126.0 (C-2′, C-6′), 114.0 (C-8), 107.5 (6-CHO), 106.6 (C-10), 79.9 (C-2), 61.8 (7-OCH₃), 44.9 (C-3), 7.1 (8-CH₃).

Compound 10 [(2S)-8-Formyl-5-hydroxy-7-methoxy-6-methylflavanone]: yellow needles; UV (MeOH) λmax: 267, 335 nm; EI-MS m/z 312 [M]⁺, 311, 235, 208, 180, 104; ¹H-NMR (CDCl₃, 500 MHz) δ 12.67 (1H, s, 5-OH), 10.21 (1H, s, 8-CHO), 7.46 (2H, m, H-2′, H-6′), 7.40 (3H, m, H-3′, H-4′, H-5′), 5.52 (1H, dd, J=12.5, 2.5 Hz, H-2), 4.03 (3H, s, 7-OCH₃), 3.01 (1H, dd, J=17.0, 12.5 Hz, H-3), 2.90 (1H, dd, J=17.0, 2.5 Hz, H-3), 2.09 (1H, s, 6-CH₃); ¹³C-NMR (CDCl₃, 125 MHz) δ 193.9 (8-CHO), 188.4 (C═O), 166.3 (C-9), 166.0 (C-7), 165.5 (C-5), 138.1 (C-1′), 128.9 (C-4′), 128.8 (C-3′, C-5′), 125.8 (C-2′, C-6′), 110.0 (C-8), 109.3 (C-6), 107.6 (C-10), 78.7 (C-2), 64.6 (7-OCH₃), 45.2 (C-3), 7.3 (6-CH₃).

Compound 13 (5,7-Dihydroxy-6,8-dimethylfavanone): yellow oil; UV (MeOH) λmax: 297, 344 nm; EI-MS m/z 284[M]⁺, 266, 207, 180, 152; ¹H-NMR (CD₃COCD₃, 500 MHz) δ 12.50 (2H, s, 5-OH, 5-OH), 7.67 (2H, d, J=8.0 Hz, H-2′, H-6′), 7.54 (3H, m, H-3′, H-4′, H-5′), 5.62 (1H, dd, J=12.5, 2.5 Hz, H-2), 3.19 (1H, dd, J=17.0, 12.5 Hz, H-3), 2.93 (1H, dd, J=17.0, 2.5 Hz, H-3), 2.15 (3H, s, 8-CH₃), 2.13 (3H, s, 6-CH₃); ¹³C-NMR (CD₃COCD₃, 125 MHz) δ 197.4 (C═O), 163.1 (C-7), 160.1 (C-5), 158.6 (C-9), 140.1 (C-1′), 129.6 (C-3′, C-5′), 129.3 (C-4′), 127.1 (C-2′, C-6′), 104.5 (C-8), 103.5 (C-6), 103.2 (C-10), 79.6 (C-2), 43.7 (C-3), 8.3 (8-CH₃), 7.6 (6-CH₃).

Compound 14 (2,2′,4′-Trihydroxy-6′-methoxy-3′,5′-dimethylchalcone): orange powder; UV (MeOH) λmax: 300, 366 nm; ¹H-NMR (CD₃COCD₃, 500 MHz) δ 8.20 (1H, d, J=16.0 Hz, H-β), 8.15 (1H, d, J=16.0 Hz, H-α), 7.67 (1H, d, J=8.0 Hz, H-6), 7.27 (1H, m, H-4), 6.97 (1H, m, H-5), 6.92 (1H, m, H-3), 3.09 (3H, s, 6′-OCH₃), 2.14 (3H, s, H-5′), 2.08 (3H, s, H-3′); ¹³C-NMR (CD₃COCD₃, 125 MHz) δ 194.2 (C═O), 163.2 (C-2′), 159.8 (C-4′), 158.7 (C-6′), 157.8 (C-2), 139.4 (C-β), 132.4 (C-4), 129.6 (C-6), 127.1 (C-α), 123.2 (C-1), 120.9 (C-5), 117.1 (C-3), 110.5 (C-1′), 109.1 (C-5′), 107.8 (C-3′), 62.6 (6′-OCH₃), 8.9 (5′-CH₃), 8.2 (3′-CH₃).

### Example 4

### Preparation of Influenza Viruses

4-1. Avian Influenza (H9N2) Virus

Avian influenza virus used in the experiment was low pathogenic avian influenza virus A/chicken/Korea/01310/2001(H9N2). This virus was inoculated into the allantoic cavity of 10-day-old SPF (Specific-Pathogen-Free) fertilized eggs, and after 2 days, collected. The collected virus was inoculated into MDCK (Madin-Darby canine kidney), cultured for 5 days and centrifuged, and the supernatant culture was used to measure the activity of viral neuraminidase of H9N2.

4-2. Swine Influenza (H1N1) Virus

Swine influenza virus used in the experiment was swine influenza virus A/Sw/Kor/CAN1/04 (H1N1, KCTC 11165BP; obtained from the Choongang Vaccine Laboratory, Korea). The virus was inoculated into the allantoic cavity of 10-day-old specific-pathogen free (SPF) eggs and, after 2 days, collected. The collected seed virus was cultured, and the cultured virus was inoculated again into the allantoic cavity of SPF eggs. The viral culture was used to measure the activity of neuraminidase of H1N1.

### Example 5

### Neuraminidase Cloning

Novel influenza virus is highly infectious, and thus when it infects people, it will have high mortality rate. For this reason, a method of expressing in an animal cell line only the neuraminidase of novel influenza virus to be targeted by a novel drug and verifying the activity of a novel drug candidate was used, rather than a method of screening and confirming an active ingredient using novel influenza virus itself. The neuraminidase gene sequence of novel influenza virus was obtained from the NCBI GenBank, and the neuraminidase of novel influenza virus was cloned. Also, a point neuraminidase mutant of novel influenza virus having resistance to Tamiflu was artificially constructed and used to investigate the activity of natural compounds. The base sequences used in the cloning are as follows:

* Neuraminidase peptide sequence of novel influenza virus used *

5-1. Neuraminidase Peptide Sequence of Novel Influenza (H1N1) Virus

MNPNQKIITIGSVCMTIGMANLILQIGNIISIWISHSIQLGNQNQIET
CNQSVITYENNTWVNQTYVNISNTNFAAGQSVVSVKLAGNSSLCPVSG
WAIYSKDNSVRIGSKGDVFVIREPFISCSPLECRTFFLTQGALLNDKH
SNGTIKDRSPYRTLMSCPIGEVPSPYNSRFESVAWSASACHDGINWLT
IGISGPDNGAVAVLKYNGIITDTIKSWRNNILRTQESECACVNGSCFT
VMTDGPSNGQASYKIFRIEKGKIVKSVEMNAPNYHYEECSCYPDSSEI
TCVCRDNWHGSNRPWVSFNQNLEYQIGYICSGIFGDNPRPNDKTGSCG
PVSSNGANGVKGFSFKYGNGVWIGRTKSISSRNGFEMIWDPNGWTGTD
NNFSIKQDIVGINEWSGYSGSFVQHPELTGLDCIRPCFWVELIRGRPK
ENTIWTSGSSISFCGVNSDTVGWSWPDGAELPFTIDK

5-2. Neuraminidase Peptide of Novel Influenza Virus Having Resistance to Tamiflu

The underlined portion is a point mutation (H→Y)

MNPNQKIITIGSVCMTIGMANLILQIGNIISIWISHSIQLGNQNQIET
CNQSVITYENNTWVNQTYVNISNTNFAAGQSVVSVKLAGNSSLCPVSG
WAIYSKDNSVRIGSKGDVFVIREPFISCSPLECRTFFLTQGALLNDKH
SNGTIKDRSPYRTLMSCPIGEVPSPYNSRFESVAWSASACHDGINWLT
IGISGPDNGAVAVLKYNGIITDTIKSWRNNILRTQESECACVNGSCFT
VMTDGPSNGQASYKIFRIEKGKIVKSVEMNAPNYYYEECSCYPDSSEI
TCVCRDNWHGSNRPWVSFNQNLEYQIGYICSGIFGDNPRPNDKTGSCG
PVSSNGANGVKGFSFKYGNGVWIGRTKSISSRNGFEMIWDPNGWTGTD
NNFSIKQDIVGINEWSGYSGSFVQHPELTGLDCIRPCFWVELIRGRPK
ENTIWTSGSSISFCGVNSDTVGWSWPDGAELPFTIDK

After gene cloning, the gene corresponding to the neuraminidase of novel influenza virus was amplified by PCR. The amplified product was cloned into a pcDNA3.1/V5-His Topo vector (Invitrogen). The cloning was confirmed by restriction enzyme cutting, and then by DNA sequencing. A neuraminidase having resistance to Tamiflu was obtained by substituting histidine with tyrosine based on literature survey. In this experiment, a PCR quick-change method was used. In this method, oligonucleotide corresponding to a portion to be substituted was constructed, and then amplified in a test tube using PCR polymerase. In order to remove a plasmid having a previous base sequence, treatment with DpnI enzyme was performed to remove only the methylated plasmid. The mutated plasmid was amplified in bacteria, and the amino acid substitution in the amplified product was confirmed by DNA sequencing.

### Example 6

### Measurement of Neuraminidase Activity

Neuraminidase activity was precisely measured using a modification of the method designed by Myers et al. A sample containing each neuraminidase was allowed to react with a mixture of 20 μl of 0.04 M sodium acetate buffer (pH 5.0) and 80 μl of 0.04 mM 4-methylumbelliferyl-α-D-N-acetylneuraminic acid (Sigma M8639) for 10 minutes, and then 100 μl of 0.1M glycine-NaOH buffer was added thereto to stop the reaction. Then, the activity of neuraminidase was measured based on a difference in fluorescence at 360 nm/440 nm using a fluorospectrophotometer. Herein, each of Tamiflu, the compounds and the solvent and fraction extracts was previously added to the cells or was added during the culture of the virus, such that the activity of neuraminidase was inhibited (when the sample is to be treated directly with the compound, 1 μl of the compound is added during the fluorescence reaction). Also, to correct the luminescence of the sample, the inhibition rate was calculated according to the following equation: wherein A: luminescence measured, B: luminescence of sample mixture, and C: luminescence of solvent in which sample is dissolved.

Inhibition rate: {C−(A−B)/C}×100,

For the kinetic study of the compounds, the concentration of 4-methoxyumbelliferone was measured before addition of 0.1M glycine-NaOH buffer for stopping the reaction. The data were analyzed using Sigmaplot 11.0 (SPCC Inc., Chicago, Ill.). Also, to measure the reversible reaction of the enzyme, a method of diluting the reaction solution containing the enzyme and the inhibitor was used.

### Example 7

### Measurement of Neuraminidase Activity Using Viral Culture

In order to measure the activity of viral neuraminidase, the activity of neuraminidase in each of the viral cultures of avian influenza virus (H9N2) and swine influenza virus was measured.

The activity of neuraminidase in the viral cultures was measured according to the method of Example 6 using the viral cultures of Examples 4-1 and 4-2. The results of measuring the activity of neuraminidase using each fraction of the ethanol extracts and compounds 1 to 14 are shown in Tables 2 and 3 below. As shown in Table 2, to measure the activities of the ethanol extracts and the solvent fractions against neuraminidase, the activity against neuraminidase of each extract (final extraction: 20 μg/Ml) in the viral cultures was measured. As can be seen from the results of Table 2, the 70% ethanol extract and the ethyl acetate fraction showed excellent activity. Also, as can be seen from the results of Table 3, the compounds of the present invention had inhibitory activity against avian influenza virus and swine influenza virus.

TABLE 2

| | Inhibitory activity | Inhibitory activity |
|---|---|---|
| | (20 μg/Ml) against | (20 μg/Ml) against |
| | neuraminidase of | neuraminidase of |
| | avian influenza | swine influenza |
| Condition | virus | virus (H1N1) |
| Fraction A (70% | 50.8% | 51.5% |
| ethanol extract) | | |
| Fraction B (hexane | — | — |
| extract) | | |
| Fraction C (ethyl | 64.5% | 60.7% |
| acetate extract) | | |
| Fraction D (butanol | 12.5% | 13.6% |
| extract) | | |

TABLE 3

| | IC₅₀ for | IC₅₀ for |
|---|---|---|
| | neuraminidase of | neuraminidase of |
| | avian influenza | swine influenza |
| Condition | virus (H9N2) | virus (H1N1) |
| Compound 1 (μg/Ml) | 115.74 ± 2.62 | 127.43 ± 2.42 |
| Compound 2 (μg/Ml) | 110.36 ± 1.97 | 112.40 ± 2.25 |
| Compound 3 (μg/Ml) | 89.42 ± 2.55 | 88.73 ± 2.07 |
| Compound 4 (μg/Ml) | 5.83 ± 0.43 | 6.42 ± 0.43 |
| Compound 5 (μg/Ml) | 6.48 ± 0.70 | 9.68 ± 0.69 |
| Compound 6 (μg/Ml) | >150 | >150 |
| Compound 7 (μg/Ml) | 107.41 ± 2.15 | 122.64 ± 2.77 |
| Compound 8 (μg/Ml) | 18.88 ± 2.33 | 24.35 ± 2.06 |
| Compound 9 (μg/Ml) | 79.95 ± 1.53 | 83.80 ± 2.97 |
| Compound 10 (μg/Ml) | 93.26 ± 2.14 | 90.46 ± 3.15 |
| Compound 11 (μg/Ml) | >150 | >150 |
| Compound 12 (μg/Ml) | >150 | >150 |
| Compound 13 (μg/Ml) | 90.63 ± 2.43 | 94.74 ± 2.63 |
| Compound 14 (μg/Ml) | 7.26 ± 0.61 | 8.83 ± 1.12 |
| Tamiflu (ng/Ml) | 4.24 ± 0.82 | 39.04 ± 1.03 |

As can be seen in Table 3, in the case of Tamiflu, the inhibitory activity (4.24±0.82 ng/Ml) against the neuraminidase of avian influenza virus was better than the inhibitory activity (39.04±1.03 ng/Ml) against the neuraminidase of swine influenza virus. Such results suggest that Tamiflu acts selectively against avian influenza virus and has low activity against swine influenza virus or novel influenza virus. However, as shown in Table 3 above, it was found that compounds 1 to 14 of the present invention inhibited both the neuraminidase of avian and swine influenza viruses at similar concentrations.

Tamiflu is a drug prepared by determining the neuraminidase protein structure of avian influenza virus and then synthesizing a compound binding to the active residue of the neuraminidase protein structure. Namely, Tamiflu is a competitive inhibitor of neuraminidase that acts on the active residue. However, recently, Tamiflu-resistant virus acquired resistance to Tamiflu by modifying the active residue on which Tamiflu acts. The present inventors examined the inhibitory mechanism of compound 4 by changing the concentration of compound 4 in order to determine the inhibitory mechanisms of chalcone-based compounds. Compound 4 that is one of the main compounds isolated from Cleistocalyx operculatus is a non-competitive inhibitor that reversibly inhibits the neuraminidase of swine influenza virus. Such results demonstrate that the calchone-based compounds non-competitively act against neuraminidase, and thus can be widely used alone or in combination with Tamiflu in spite of the appearance of Tamiflu-resistant virus.

### Example 8

### Measurement of Activity of Neuraminidase in Lysed Cell Solution Directly Expressing Neuraminidase

In order to measure the activity of neuraminidase in a lysed cell solution directly expressing the neuraminidases of novel influenza virus and Tamiflu-resistant novel influenza virus, human kidney HEK293T cells were treated with 0.25% trypsin, and the supernatant was removed, and the remaining cells were suspended in FBS-free DMEM medium. Then, 3 μl of lipofectamin (Invitrogen, Inc.) in 100 μl of FBS-free DMEM was dropped into the cell suspension, and then shaken slowly and allowed to stand at room temperature for 15 minutes. Then, in order to transfect plasmids expressing the neuraminidase of novel influenza virus (Example 5-1) and the neuraminidase of Tamiflu-resistant novel influenza virus (Example 5-2), 1 μg of each of the plasmids was dropped slowly into a micro-centrifuge tube over 30 seconds and allowed to stand at room temperature for 15 minutes. The lipofectamin/DNA-plasmid mixture and the HEK293T cell line were mixed slowly, and the resulting suspension was incubated at room temperature for 20 minutes. Then, the suspension was incubated at room temperature for 20 minutes, after which it was seeded in a 35-mm culture dish and incubated in a CO₂ incubator for 24 hours. Then, the HEK293T cells were washed twice with PBS, and 500 μl of each of the extracts was added to the culture dish to lyse the cells, followed by centrifugation at 14,000 rpm for 5 minutes. The obtained supernatant was used to test neuraminidase activity according to the method of Example 6.

Table 4 below shows the results of measuring the activities of compounds 4, 5, 8 and 14 (among compounds 1 to 14 isolated from the Cleistocalyx operculatus extract) against the neuraminidases of novel influenza virus and Tamiflu-resistant novel influenza virus).

As can be seen in Table 4, compounds 4, 5, 8 and 14 had excellent inhibitory activity not only against the neuraminidase of novel influenza virus, but also against the neuraminidase of Tamiflu-resistant novel influenza virus. On the other hand, Tamiflu showed an IC₅₀ value of 21.13±1.36 ng/Ml against the neuraminidase of novel influenza virus, but showed an IC₅₀ of 5.04±0.41 μg/Ml against the neuraminidase of Tamiflu-resistant novel influenza virus. This suggests the risk of the neuraminidase of Tamiflu-resistant novel influenza virus and also indicates that compounds 4, 5, 8 and 14 of the present invention can be used against the neuraminidase of novel influenza virus and the neuraminidase of Tamiflu-resistant novel influenza virus.

TABLE 4

| | | IC₅₀ against |
|---|---|---|
| | IC₅₀ against | neuraminidase of |
| | neuraminidase of novel | Tamiflu-resistant |
| Condition | influenza virus | novel influenza virus |
| Compound 4 (μg/Ml) | 2.56 ± 0.33 | 1.04 ± 0.42 |
| Compound 5 (μg/Ml) | 10.50 ± 0.82 | 1.50 ± 0.26 |
| Compound 8 (μg/Ml) | 26.63 ± 1.52 | 7.39 ± 0.86 |
| Compound 14 (μg/Ml) | 7.39 ± 0.86 | 0.80 ± 0.09 |
| Tamiflu | 21.13 ± 1.36 ng/Ml * | 5.04 ± 0.41 μg/Ml * |

* the difference in IC₅₀ of Tamiflu against novel influenza virus from IC₅₀ against resistant virus corresponds to a difference between ng/Ml and μg/Ml

### Example 9

### Measurement of Activity of Tamiflu in Combination with Compound 4 Against Neuraminidase in Viral Culture

As shown in Example 7, the IC₅₀ of Tamiflu was 39.04±1.03 ng/Ml against the neuraminidase of swine influenza virus and 4.24±0.82 ng/Ml against the neuraminidase of avian influenza virus, suggesting that Tamiflu is a competitive inhibitor. In comparison with this, compound 4 that is one of the compounds of the present invention was a noncompetitive inhibitor having an IC₅₀ of 6.42±0.43 μg/Ml against swine influenza virus.

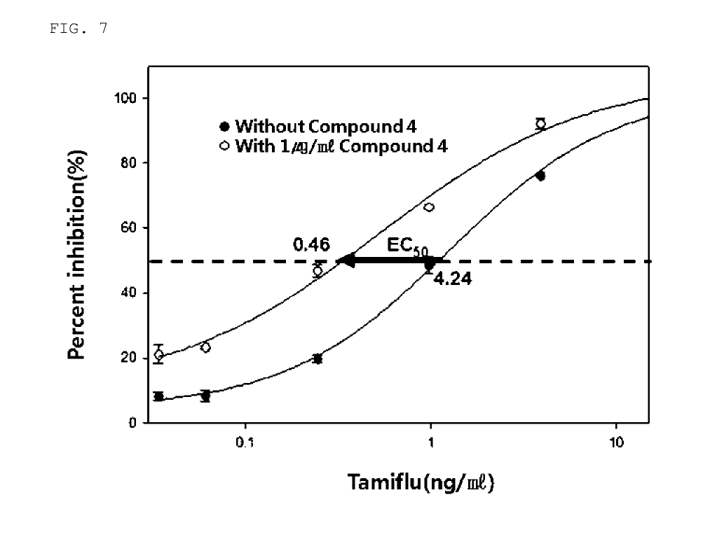

A competitive inhibitor acts in an active pocket in which the neuraminidase enzyme binds with the compound, and thus if compound 4 that acts in other sites of the neuraminidase enzyme is added, the activity of Tamiflu can be increased. Under this assumption, compound 4 was used to calculate the IC₅₀ of Tamiflu. For this purpose, the concentration of compound 4 derived from Cleistocalyx operculatus was fixed to 1 μg/Ml, and then the IC₅₀ values of Tamiflu against the neuraminidase of swine influenza virus and avian influenza virus were measured using the viral cultures of Examples 4-1 and 4-2 according to the method of Example 6. As a result, as shown in FIGS. 6 and 7, the IC₅₀ value of Tamiflu against swine influenza virus was 10.51±0.64 ng/Ml, suggesting that the use of Tamiflu in combination with compound 4 could increase the activity of Tamiflu by about 3.71 times. Also, the IC₅₀ value of Tamiflu against avian influenza virus was 0.46±0.42 ng/Ml, suggesting that the use of Tamiflu in combination with compound 4 could increase the activity of Tamiflu by about 9.22 times. For novel influenza or avian influenza pandemic, the prescription of Tamiflu alone, Tamiflu+Amantadine (mainly rimantidine) or zanamivir is currently recommended. However, Amantadine has high toxicity. Thus, a combination therapy of the compound of the present invention and Tamiflu is an effective strategy for the treatment of diseases caused by resistant viruses.

### Example 10

### Toxicity Test

10-1. Acute Toxicity

In order to examine the acute toxicity of the 70% ethanol extract and the ethyl acetate fraction (derived from Cleistocalyx operculatus) in animals within 24 hours after the ethanol extract and the ethyl acetate fraction were administered to the animals in excess amounts within a short time, and also to determine the mortality thereof, the following test was carried out. For this purpose, 20 ICR mice were allotted into two groups: a control group of 10 animals, and a test group of 10 animals. The control group was administered only with a solvent, and the test group was administered orally with the ethyl acetate active fraction extracted from Cleistocalyx operculatus at a concentration of 4.0 g/kg (about 100 times the amount used in general animal tests). 24 hours after the administration, the mortality of each group was examined. As a result, in the control group and the test group (administered with each of the 70% ethanol extract of Cleistocalyx operculatus and the ethyl acetate active fraction containing compounds 1 to 14), all the animals survived.

10-2. Toxicity test for organ and tissue of test group and control group

In an organic toxicity test, in order to examine the effect of the extract the present invention on the organ (tissue) of C57BL/6J mice, blood was collected from the animals of each of the test group (administered with the 70% ethanol extract containing compounds 1 to 14) and the control group (administered only with the solvent) 8 weeks after the administration. The levels of GPT (glutamate-pyruvate transferase) and BUN (blood urea nitrogen) in the collected blood were measured. As a result, GPT known to have a connection with liver toxicity and BUN known to have a connection with kidney toxicity showed no significant difference between the control group and the test group. Also, livers and kidneys were collected from the animals, and tissue sections were prepared from the collected organs according to a conventional method. The tissue sections were histologically observed with an optical microscope, and as a result, no special abnormality was observed.

### Use Example 1

### Pharmaceutical Preparation>

1-1. Preparation of Tablets

200 g of each of the 70% ethanol extract of Cleistocalyx operculatus according to Example 1 of the present invention and the compounds isolated from the extract was mixed with 175.9 g of lactose, 180 g of potato starch and 32 g of colloidal silicic acid. 10% gelatin solution was added to the mixture, which was then ground and sieved through a 14-mesh screen. The sieved material was dried and 160 g of potato starch, 50 g of talc and 5 g of magnesium stearate were added thereto.

The mixture was compressed into tablets.

1-2. Preparation of Injectable Solution

1 g of each of the 70% ethanol extract of Cleistocalyx operculatus according to Example 1 of the present invention and the compounds isolated from the extract was dissolved in distilled water together with 0.6 g of sodium chloride and 0.1 g of ascorbic acid to make 100 ml of a solution. The solution was bottled and sterilized by heating at 20° C. for 30 minutes.

### Use Example 2

### Food Preparation

2-1. Preparation of Seasoning for Cooking

Each of the 70% ethanol extract of Cleistocalyx operculatus according to Example 1 of the present invention and the compounds isolated from the extract was used to prepare a cooking seasoning for health promotion containing the ethanol extract or compound in an amount of 0.2-10 wt %.

2-2. Preparation of Wheat Flour Food

Each of the 70% ethanol extract of Cleistocalyx operculatus according to Example 1 of the present invention and the compounds isolated from the extract was added to wheat flour in an amount of 0.1-5.0 wt %, and the mixture was used to prepare foods for health promotion, including bread, cakes, cookies, crackers and noodles.

2-3. Preparation of Soups and Gravies

Each of the 70% ethanol extract of Cleistocalyx operculatus according to Example 1 of the present invention and the compounds isolated from the extract was added to soup or gravy in an amount of 0.1-1.0 wt %, thereby preparing processed meat products, noodle soups and gravies.

2-4. Preparation of Dairy Products

Each of the 70% ethanol extract of Cleistocalyx operculatus according to Example 1 of the present invention and the compounds isolated from the extract was added to milk in an amount of 0.1-1.0 wt %, and the milk was used to prepare various dairy products such as butter and ice cream.

### Use Example 3

### Beverage Preparation

3-1. Preparation of Vegetable Juice

0.5 g of each of the 70% ethanol extract of Cleistocalyx operculatus according to Example 1 of the present invention and the compounds isolated from the extract was added to 1000 ml of tomato or carrot juice, thereby preparing vegetable juice for health promotion.

3-2. Preparation of Fruit Juice

0.1 g of each of the 70% ethanol extract of Cleistocalyx operculatus according to Example 1 of the present invention and the compounds isolated from the extract was added to 1000 ml of apple or grape juice, thereby preparing fruit juice for health promotion.

### Use Example 4

### Preparation of Feed Additives

10.0 g of each of the 70% ethanol extract of Cleistocalyx operculatus according to Example 1 of the present invention and the compounds isolated from the extract was added to 50.0 g of a carrier, thereby preparing feed additives. However, the mixing ratio can be changed and a feed additive may also be prepared using the above components according to a conventional method for preparing feed compositions.

### Use Example 5

### Preparation of Disinfectant

3.0 g of each of the 70% ethanol extract of Cleistocalyx operculatus according to Example 1 of the present invention and the compounds isolated from the extract was added to 1000 ml of purified water, thereby preparing a preservative-free natural disinfectant.

What is claimed is:

1. A Cleistocalyx operculatus extract for preventing or treating avian and swine influenza- or novel influenza-related diseases, in which the extract is obtained by extracting Cleistocalyx operculatus with at least one extraction solvent selected from the group consisting of ethanol, methanol, and aqueous solutions thereof, and the extract containing at least one compound selected from the group consisting of 7-hydroxy-5-methoxy-6,8-dimethylisoflavone; 5,7-dihydroxy-6,8-dimethyldihydroflavonol; 2,7-dihydroxy-5-methoxy-6,8-dimethylflavanone; 4,2′,4′-trihydroxy-6′-methoxy-3′,5′-dimethylchalcone; 2′,4′-dihydroxy-6′-methoxy-3′,5′-dimethylchalcone; 7-hydroxy-5-methoxy-6,8-dimethylfavanone; 2′,4′-dihydroxy-3′-methyl-6′-methoxychalcone; 6-formyl-8-methyl-7-O-methylpinocembrin; (2S)-8-formyl-5-hydroxy-7-methoxy-6-methylflavanone; 5,7-dihydroxy-6,8-dimethylfavanone; and 2,2′,4′-trihydroxy-6′-methoxy-3′,5′-dimethylchalcone.

2. The Cleistocalyx operculatus extract of claim 1, wherein the extraction solvent is a 50-100% ethanol aqueous solution.

3. A health functional food for preventing or alleviating avian and swine influenza- or novel influenza-related diseases, comprising the Cleistocalyx operculatus extract of claim 1.

4. The health functional food of claim 1, wherein the health functional food is selected from the group consisting of dairy products, including drinks, meats, sausages, bread, candies, snacks, noodles, and ice creams; beverages, including soups and ion beverages, and nutrition supplement products, including alcoholic beverages or vitamin complexes.

5. A pharmaceutical composition for preventing or treating avian and swine influenza- or novel influenza-related diseases, comprising the Cleistocalyx operculatus extract of claim 1 as an active ingredient together with a pharmaceutically acceptable carrier or excipient.

6. A pharmaceutical composition for preventing or treating avian and swine influenza- or novel influenza-related diseases, comprising at least one of the following compounds: 7-hydroxy-5-methoxy-6,8-dimethylisoflavone; 5,7-dihydroxy-6,8-dimethyldihydroflavonol; 2,7-dihydroxy-5-methoxy-6,8-dimethylflavanone; 4,2′,4′-trihydroxy-6′-methoxy-3′,5′-dimethylchalcone; 2′,4′-dihydroxy-6′-methoxy-3′,5′-dimethylchalcone; 7-dydroxy-5-methoxy-6,8-dimethylfavanone; 2′,4′-dihydroxy-3′-methyl-6′-methoxychalcone; 6-formyl-8-methyl-7-O-methylpinocembrin; (2S)-8-formyl-5-hydroxy-7-methoxy-6-methylflavanone; 5,7-dihydroxy-6,8-dimethylfavanone; and 2,2′,4′-trihydroxy-6′-methoxy-3′,5′-dimethylchalcone.

7. The pharmaceutical composition of claim 6, wherein the composition has inhibitory activities against the neuraminidase of avian influenza virus, the neuraminidase of novel influenza virus and the neuraminidase of Tamiflu-resistant novel influenza virus.

8. A pharmaceutical composition for preventing or treating avian and swine influenza- or novel influenza-related diseases, comprising the compound of claim 6 and Tamiflu as active ingredients together with a pharmaceutically acceptable carrier or excipient.

9. An animal drug and feed additive for preventing or treating avian and swine influenza- or novel influenza-related diseases, comprising the Cleistocalyx operculatus extract of claim 1 as an active ingredient together with a pharmaceutically acceptable carrier or excipient.

10. A natural disinfectant for preventing or treating avian and swine influenza- or novel influenza-related diseases, comprising the Cleistocalyx operculatus extract of claim 1 as an active ingredient together with a pharmaceutically acceptable carrier or excipient.

11. 7-Hydroxy-5-methoxy-6,8-dimethylisoflavone isolated from Cleistocalyx operculatus .

12. 5,7-Dihydroxy-6,8-dimethyldihydroflavonol isolated from Cleistocalyx operculatus .

13. 2,7-Dihydroxy-5-methoxy-6,8-dimethylflavanone isolated from Cleistocalyx operculatus .

14. 4,2,4′-Trihydroxy-6′-methoxy-3′,5′-dimethylchalcone isolated from Cleistocalyx operculatus.


SEQUENCE LISTING (2 sequences)

SEQ ID NO: 1 - protein, 469 aa, swine influenza virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 2 - protein, 469 aa, swine influenza virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYYYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK